(19) United States
(12) Patent Application Publication
(10) Pub. No.: US 2009/0013787 A1
(43) Pub. Date: Jan. 15, 2009

(54) Hearing testing device

(76) Inventor: Philip Stuart Esnouf, Richmond (AU)

Correspondence Address: SEED INTELLECTUAL PROPERTY LAW GROUP PLLC, 701 FIFTH AVE, SUITE 5400, SEATTLE, WA, 98104, US

(21) Appl. No.: 11/547,585

(22) PCT Filed: Apr. 8, 2005
(86) PCT No.: PCT/AU2005/000517

§ 371 Date: Sep. 30, 2008

(30) Foreign Application Priority Data
Apr. 8, 2004 (AU) 2004901920

(51) Int. Cl. A61B 5/12; H04R 29/00

(52) U.S. Cl. 73/585; 381/56; 600/559

(57) ABSTRACT

A hearing testing device (2) for self-testing the hearing of a subject including: an earpiece housing (4); a loud speaker (46) located within the housing; and control circuitry (14) coupled to drive the loudspeaker, the control circuitry being arranged to produce a sequence of test signals which are inputted to the loudspeaker to produce a series of tones at frequencies corresponding to said test signals wherein said test signals do not include significant harmonic components.

This invention relates to a hearing testing device and a method of assessing hearing impairment of a subject.

More particularly, the invention relates to a hearing testing device which can be used by a subject in order to measure, with a reasonable level of accuracy, whether a subject's hearing is impaired at predetermined frequencies. The device is intended to be used as an initial screening only which is quick and easy to use. If the user or his or her medical practitioner detects a problem then the subject can be referred to hearing centres which have specialised equipment for accurately measuring and detecting hearing impairment of a subject.

According to the present invention there is provided a hearing testing device for self-testing the hearing of a subject including:

an earpiece housing;

a loudspeaker located within the housing; and

control circuitry coupled to drive the loudspeaker, the control circuitry being arranged to produce a sequence of test signals which are inputted to the loudspeaker to produce a series of tones at frequencies corresponding to said test signals.

Preferably further, the control circuitry produces test signals which are sinusoidal.

Preferably the test signals produce sounds which have a total harmonic distortion below a predetermined level. Preferably the distortion is less than 5% and most preferably about 3%.

Preferably further, the level is about 20 to 23 dB. Preferably the series of sounds are produced at 500 Hz, 1000 Hz, 1500 Hz, 2000 Hz and 3000 Hz.

Preferably further, the sounds are separated by pauses.

Preferably, the duration of the pauses is in the range 1 to 2 seconds and most preferably 1.5 seconds.

Preferably, the duration of the sounds is in the range 1 to 2 seconds and most preferably 1.5 seconds.

Preferably further, the device includes a visual indicator to indicate to the user when a tone is being produced.

In the preferred form of the invention, the device is portable and is battery powered. In this case the circuitry needs to be compact so that it can conveniently fit inside the earpiece housing.

Compact circuitry is available which can produce square waves or other pulse waveforms which can be modulated to produce signals at the aforementioned frequencies. If, however, pulse or square wave signals were inputted to the loudspeaker, there would be substantial harmonic content and/or harmonic distortion which could lead to the subject being able to falsely discern a sound at a particular frequency when in fact the subject heard only the harmonic component rather than the test frequency. For instance, at 500 Hz the subject may have no perceptible hearing of the test signal but may falsely believe that a sound is heard at this level when in fact the subject is hearing a harmonic at say 1000 Hz or above.

A specific object of preferred embodiments of the invention is to be able to produce compact circuitry which can produce test signals which are substantially pure sinusoidal waves so as to eliminate or substantially eliminate false readings attributable to harmonics and/or harmonic distortion.

The invention also provides a method of assessing hearing impairment of a subject including the steps of:

locating a portable earpiece having a loudspeaker over an ear of the subject;

actuating a switch which is coupled to control circuitry in the earpiece to generate a sequence of test signals;

inputting the test signals to the loudspeaker which, in response thereto, generates a series of tones which do not include significant harmonic components; and

determining whether the subject can hear each of said tones.

The invention will now be further described with reference to the accompanying drawings, in which:

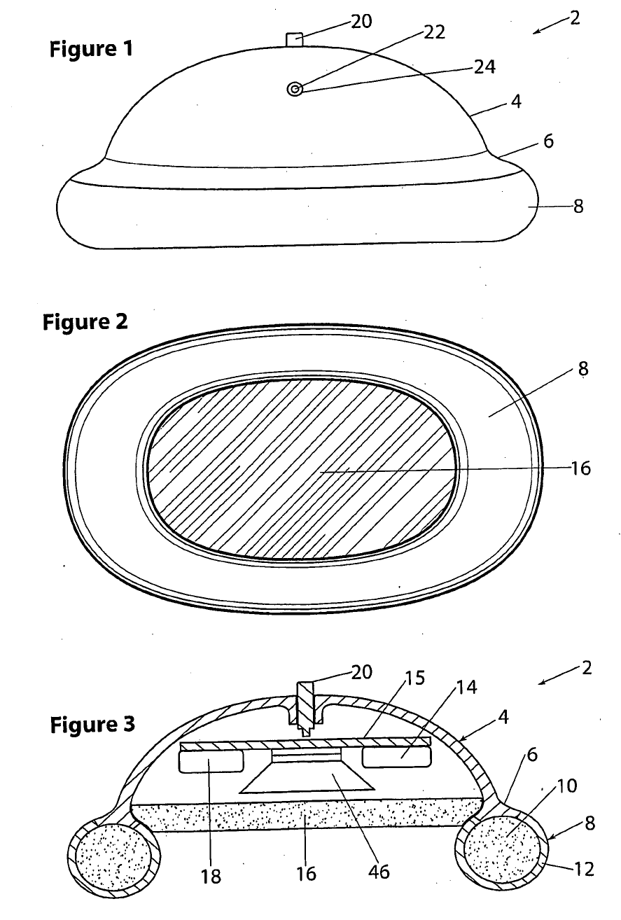

FIG. 1 is a side view of a hearing device of the invention;

FIG. 2 is a plan view of the hearing device of the invention;

FIG. 3 is a cross-sectional view through the hearing device of the invention;

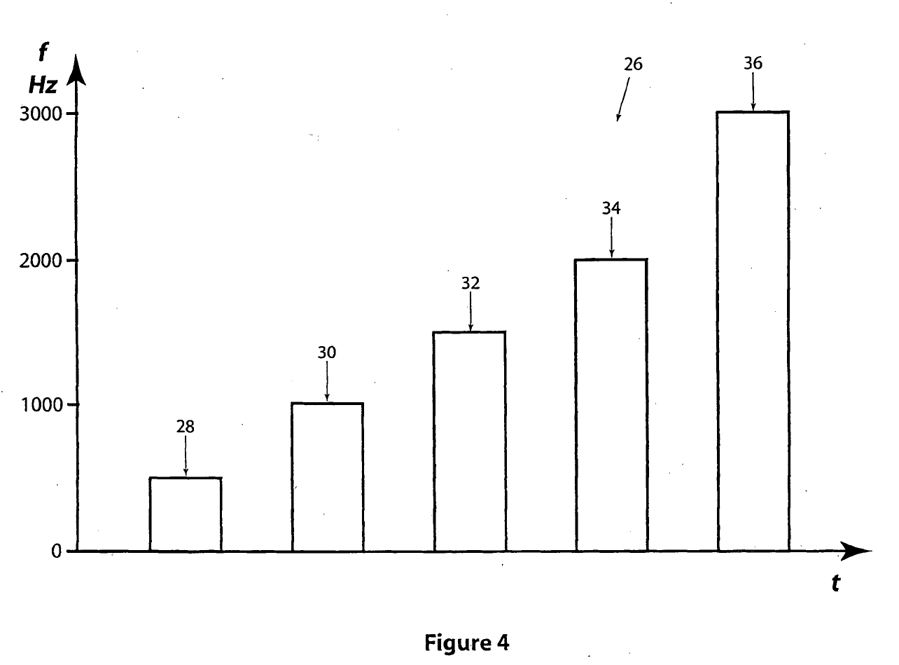

FIG. 4 is a graphical representation of the output of the device of the invention;

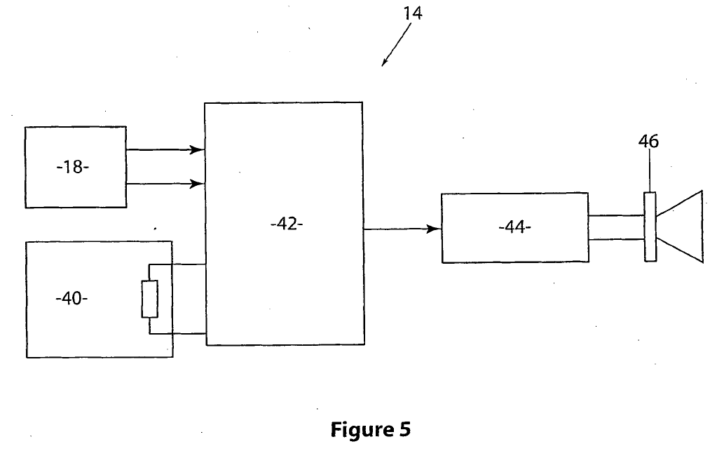

FIG. 5 is a schematic diagram for the audio components of the device; and

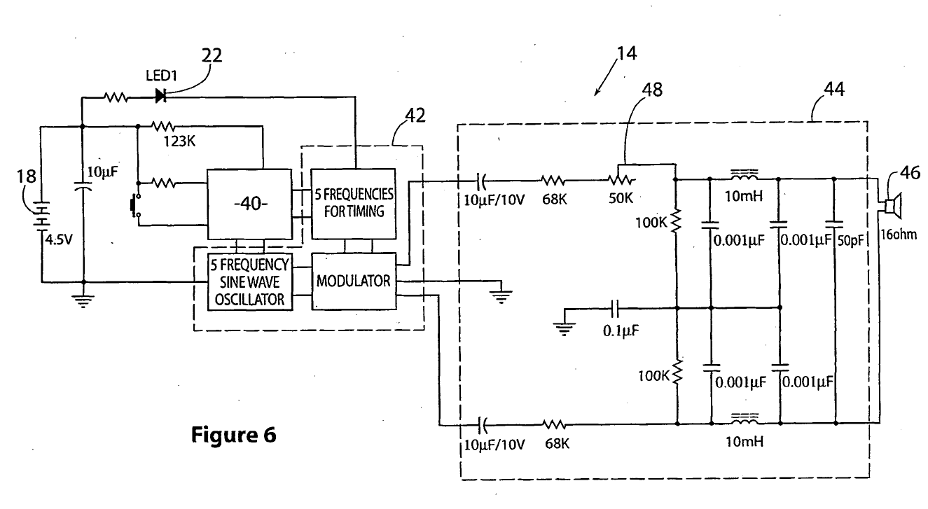

FIG. 6 is a detailed circuit diagram for the audio components of the device.

FIG. 1 shows a hearing testing device 2 constructed in accordance with the invention. It includes a hollow shell-like housing 4 which is cup shaped and preferably moulded from plastics material. The housing has a lower flared portion 6 to which is connected a padded ring 8. As best seen in FIG. 3, the padded ring 8 includes a core 10 of foam material covered by a pliable skin 12. As best seen in FIG. 3, tone generating circuitry 14 is mounted on a circuit board 15 located within the interior of the housing 4. A layer 16 of foam material is located across the open face of the housing so as to prevent dirt and foreign objects interfering with the tone generating circuitry 14. The layer 16 can be removed to provide access to batteries 18 which power the tone generating circuitry 14.

The device includes a spring-loaded press button 20 which can be pressed by a user in order to commence the testing sequence. The press button 20 is coupled to a switch 21 which is mounted on the circuit board 15 (as shown in FIG. 6). The device 2 includes an indicator LED 22 for indicating when sounds are produced by the tone generating circuitry 14. In the preferred form of the invention, the tone generating circuitry is coupled to an LED 22 and the housing 4 includes an opening 24 to enable a doctor or supervisor to see whether or not the LED 22 is or is not illuminated. A user could also use the device in front of a mirror where the user performs a self test. The LED is optional because the user will know there is a problem if five tones are not heard.

The tone generating circuit 14 is coupled to the switch 20 and after the switch 20 is pressed, is arranged to produce a sequence of tones which are substantially sinusoidal separated by pauses. The LED 22 is illuminated at the same time the sounds are produced.

In the preferred form of the invention, the circuitry 14 produces a sequence which includes bursts of substantially pure sinusoidal sounds at 500 Hz, 1000 Hz, 1500 Hz, 2000 Hz and 3000 Hz. In the preferred form of the invention, each of the sounds has a duration in the range from 1 to 2 seconds and preferably 1.5 seconds separated by pauses, each having a duration of 1 to 2 seconds and preferably 1.5 seconds. The total harmonic distortion of the sounds is preferably less than 5% and most preferably about 3%.

Preferably further, the amplitude of the sounds produced by the circuit 14 are in the range 22 to 25 dB as measured in the vicinity of the layer 16.

FIG. 4 diagrammatically shows the sequence 26 of sounds having a first burst 28 at 500 Hz followed by a pause; a second burst 30 at 1000 Hz followed by a pause; a third burst 32 at 1500 Hz followed by a pause; a fourth burst 34 at 2000 Hz followed by a pause and finally a fifth burst 36 at 3000 Hz.

In use, a user holds the device adjacent to his or her ear such that the ear is located within the padded ring 8, the padded ring being pressed against the head of the user so as to substantially screen out ambient sounds. The user then presses the switch 20 in order to commence operation of the sequence 26. The user is instructed to note how many different sounds are heard. If the user hears five sounds then the screening test indicates that the hearing is satisfactory. If, however, less than five sounds are heard then there is hearing impairment at one or more of the frequencies.

FIG. 5 diagrammatically illustrates the tone generating circuit 14. The circuitry includes the battery 18, and an oscillator 40 which produces a pulsed or square waveform clocking signal at a predetermined frequency, for example, at 6 kHz. The oscillator 40 is coupled to audio processing circuitry 42. The processing circuitry 42 is driven by the clocking signal from the oscillator 40 to generate bursts of sinusoidal signals at 500 Hz, 1000 Hz, 1500 Hz, 2000 Hz and 3000 Hz as in the sequence shown in FIG. 4. Output from the processing circuitry 42 passes to a compensating circuit 44 and then to a loudspeaker 46. The loudspeaker 46 could be of conventional design or any sound producing transducer.

The loudspeaker 46 produces sounds which correspond to the frequencies of the sequence of test signals produced by the processing circuitry 42. As with most loudspeakers, the frequency response is not perfect, that is to say the amplitude will normally show some variation with frequency. The compensating circuit 44 is essentially a filter which negates the imperfect frequency response of the loudspeaker 46 so that it produces substantially the same amplitude at each of the tones it produces.

FIG. 6 is a more detailed circuit diagram for the tone generating circuit 14. It will be seen that the light emitting diode (LED) 22 is coupled to the processing circuit 42 and is arranged so that it will only be illuminated when the sequence of tone signals is produced. Preferably, the LED 22 is illuminated only during the time when a tone is being produced.

The processing circuit 42 shown in FIG. 6 includes an oscillator module 40, a timer module 50, a sine wave generator module 52 and a modulator module 54. The oscillator module 40 generates a clocking signal, as described above, preferably at 6 kHz. The present invention is able to work with a clocking signal of any frequency. The sine wave generator module 52 is coupled to memory (not shown in FIG. 6), such as any kind of read-only memory (ROM) or random-access memory (RAM) module. The memory stores data signals corresponding to several different sine wave signals, each corresponding to a different frequency. For example, the data signals correspond to at least one complete cycle of each of 500 Hz, 1000 Hz, 1500 Hz, 2000 Hz and 3000 Hz sine wave signals. Preferably, each data signal stored in memory corresponds to a sampling rate that ranges between and including 1 kbps (kilobits per second) and 1 mbps (megabits per second). In the preferred embodiment, all the data signals stored in memory are sampled at 240 kbps.

The sine wave generator module 52 retrieves a sine wave signal of a particular frequency from memory at intervals determined using the clocking signal from the oscillator module 40. For example, if a 3 kHz signal is to be produced using a 6 kHz clocking signal, the sine wave generator module 52 retrieves a signal from memory corresponding to one cycle of a 3 kHz sine wave and outputs the same 3 kHz signal to the modulator module 54 every 2 cycles of the clocking signal. Similarly, if a 2 kHz signal is to be produced using a 6 kHz clocking signal, the sine wave generator module 52 retrieves a signal from memory corresponding to one cycle of a 2 kHz sine wave and outputs the same 2 kHz signal to the modulator module 54 every 3 cycles of the clocking signal. This process can be repeated for each of the different frequencies to be generated, as shown by way of example in Table 1:

TABLE 1

| Frequency of | Frequency of the | Delay (in no. of cycles |
|---|---|---|
| the clocking | sine wave to be | of the clocking signal) |
| signal (kHz) | generated (kHz) | between each signal output |
| 6 | 0.5 | 12 |
| 6 | 1 | 6 |
| 6 | 1.5 | 4 |
| 6 | 2 | 3 |
| 6 | 3 | 2 |

It will be apparent that the number of delay cycles corresponding to each different frequency of sine wave to be generated by the sine wave generator module 52 is to be adjusted according to the frequency of the clocking signal used, and/or according to the number of sine wave cycles for that frequency stored in memory.

The timer module 50 triggers the sine wave generator module 52 to generate a particular tone, starting from the first tone, and also determines the duration of a tone produced and the duration of silence between each tone based on the clocking signal from the oscillator module 40. The timer module 50 controls the modulator module 54 either to pass the output produced by the sine wave generator module 52 to the compensating circuit 44 (i.e. the “on” state), or to not pass any signal from the sine wave generator module 52 to the compensating circuit 44 at all (i.e. the “off” state). For example, if a clocking signal of 6 kHz is used, and the tone duration and the duration of silence are both 1.5 seconds, the timer module 50 triggers the sine wave generator module 52 to generate a tone, turn the modulator module 54 “on” for 9000 cycles of the clocking signal, and then turn the modulator module 52 “off” for a further 9000 cycles of the clocking signal. This process is then repeated for another tone of a different frequency. As discussed above, the preferred embodiment generates tones represented by a 500 Hz, 1000 Hz, 1500 Hz, 2000 Hz and 3000 Hz sine wave signal in that order.

One circuit realisation for the compensating circuit 44 is also shown in FIG. 6. This circuit functions as a filter and its operation would be understood by those skilled in the art. The circuit includes a variable resistor 48, the value of which can be adjusted at the time of manufacture so as to control the characteristics of the filter so that the amplitude of the tone signals are applied to the loudspeaker 46 such that the output from the loudspeaker produces sounds which range in amplitude from 22 dB to 25 dB.

As will be understood by those skilled in the art, the processes executed by the modules in the processing circuit 42 can be performed entirely using software, can also be executed at least in part by dedicated hardware circuits, e.g., Application Specific Integrated Circuits (ASICs). In one convenient circuit embodiment, it is possible to utilise an integrated voice synthesiser circuit in order to produce the required tone signals. One suitable circuit is an SLS603 circuit manufactured by Sun Link Technology Corporation.

In an alternative embodiment provision can be made for generation of the sequence of tones at a sound level which is different from the initial sound level. For instance, the user may operate a switch (not shown) which is operable to selectively cause the circuitry 14 to generate tones which are ±2 dB above or below the initial volume level. The switch can be manually selectable or could be electronic so that the 2 dB change in volume level occurs automatically following generation of the initial tone sequence.

Many modifications will be apparent to those skilled in the art without departing from the spirit and scope of the invention.

1. A hearing testing device for self-testing the hearing of a subject including:
an earpiece housing;
a loudspeaker located within the housing; and
control circuitry coupled to drive the loudspeaker, the control circuitry being arranged to produce a sequence of test signals which are inputted to the loudspeaker to produce a series of tones at frequencies corresponding to said test signals wherein said test signals do not include significant harmonic components.

2. A device as claimed in claim 1 wherein the control circuitry produces test signals which are sinusoidal.

3. A device as claimed in claim 1 or 2 wherein the control circuitry produces test signals which have a total harmonic distortion less than 5%.

4. A device as claimed in claim 3 wherein the total harmonic distortion is less than 3%.

5. A device as claimed in any one of claims 1 to 4 wherein the series of test signals includes signals approximately 500 Hz, 1000 Hz, 1500 Hz, 2000 Hz and 3000 Hz.

6. A device as claimed in any one of claims 1 to 5 wherein the control circuitry includes:
sound processing circuitry which in use produces said series of test signals, each test signal having substantially the same amplitude and a compensating circuit coupled between the sound processing circuitry and the loudspeaker, the compensating circuit being operable to adjust the amplitudes of the test signals so that the series of tones generated by the loudspeaker having substantially the same amplitude.

7. A device as claimed in any one of claims 1 to 6 wherein the amplitude of the series of tones is 20 to 23 dB.

8. A device as claimed in any one of claims 1 to 7 wherein the control circuitry produces pauses of predetermined duration between the test signals.

9. A device as claimed in claim 8 wherein the pauses are of the same duration.

10. A device as claimed in claim 9 wherein the duration of the pause is in the range 1 to 2 seconds.

11. A device as claimed in claim 9 or 10 wherein the duration of each test signal is the same.

12. A device as claimed in claim 11 wherein the duration of each test signal is in the range 1 to 2 seconds.

13. A device as claimed in claim 12 wherein the duration of each pause and each test signal is 1.5 seconds.

14. A device as claimed in any one of claims 1 to 13 wherein the device includes a visual indicator to indicate to the user when a tone is being produced.

15. A device as claimed in any one of claims 1 to 14 wherein the housing includes a cup shaped body having a rim and a padded ring extending about the rim.

16. A device as claimed in claim 15 wherein a layer of foam material spans the cup shaped body at or near the rim to conceal the loudspeaker and control circuitry.

17. A device as claimed in claim 15 or 16 wherein the control circuitry includes a switch which can be operated by a laser to cause the control circuitry to produce said sequence of test signals.

18. A device as claimed in claim 17 wherein the switch is coupled to an actuator which is mounted on the cup shaped housing.

19. A device as claimed in claim 18 wherein the actuator is a spring loaded press button.

20. A device as claimed in any one of claims 1 to 19 wherein the device is hand held and/or portable.

21. A method of assessing hearing impairment of a subject including the steps of:
locating a portable earpiece having a loudspeaker over an ear of the subject;
actuating a switch which is coupled to control circuitry in the earpiece to generate a sequence of test signals;
inputting the test signals to the loudspeaker which, in response thereto, generates a series of tones which do not include significant harmonic components; and
determining whether the subject can hear each of said tones.
